(19) United States
(12) Patent Application Publication
(10) Pub. No.: US 2024/0277298 A1
(43) Pub. Date: Aug. 22, 2024

(54) PHYSIOLOGICAL PARAMETER SYSTEM

(71) Applicant: Masimo Corporation, Irvine, CA (US)

(72) Inventors: Ammar Al-Ali, San Juan Capistrano, CA (US); John Graybeal, Grantville, PA (US); Massi Joe E. Kiani, Laguna Niguel, CA (US); Michael Petterson, Dana Point, CA (US)

(21) Appl. No.: 18/584,637

(22) Filed: Feb. 22, 2024

Related U.S. Application Data
(63) Continuation of application No. 17/466,287, filed on Sep. 3, 2021, now Pat. No. 11,937,949.
Application No. 17/466,287 is a division of application No. 16/159,278, filed on Oct. 12, 2018, now Pat. No. 11,109,814.
Application No. 16/159,278 is a continuation of application No. 14/275,525, filed on May 12, 2014, now Pat. No. 10,098,591.
Application No. 14/275,525 is a division of application No. 12/188,154, filed on Aug. 7, 2008, now Pat. No. 8,721,542.
Application No. 12/188,154 is a continuation of application No. 11/075,389, filed on Mar. 8, 2005, now Pat. No. 7,415,297.
(60) Provisional application No. 60/551,165, filed on Mar. 8, 2004.
(60) Provisional application No. 60/600,640, filed on Aug. 11, 2004.

(51) Int. Cl. A61B 5/00; A61B 5/083; A61B 5/1455

(52) U.S. Cl. CPC A61B 5/7275; A61B 5/14551; A61B 5/7221; A61B 5/746; A61B 5/0836; A61B 2560/0276

(57) ABSTRACT

A physiological parameter system has one or more parameter inputs responsive to one or more physiological sensors. The physiological parameter system may also have quality indicators relating to confidence in the parameter inputs. A processor is adapted to combine the parameter inputs, quality indicators and predetermined limits for the parameters inputs and quality indicators so as to generate alarm outputs or control outputs or both.

### CROSS-REFERENCE TO RELATED APPLICATIONS

This application is a continuation of U.S. patent application Ser. No. 17/466,287, titled Physiological Parameter System filed Sep. 3, 2021, which is a divisional of U.S. patent application Ser. No. 16/159,278, titled Physiological Parameter System, filed Oct. 12, 2018, which is a continuation of U.S. patent application Ser. No. 14/275,525 (now U.S. Pat. No. 10,098,591), titled Physiological Parameter System, filed May 12, 2014, which is a divisional of U.S. patent application Ser. No. 12/188,154 (now U.S. Pat. No. 8,721,542), titled Physiological Parameter System, filed Aug. 7, 2008, which is a continuation of U.S. patent application Ser. No. 11/075,389 (now U.S. Pat. No. 7,415,297), titled Physiological Parameter System, filed Mar. 8, 2005, which relates to and claims the benefit of U.S. Provisional Applications No. 60/551,165, titled Combined Physiological Parameter Monitor, filed Mar. 8, 2004 and No. 60/600,640, titled Physiological Parameter Controller, filed Aug. 11, 2004. Each of the foregoing applications are incorporated by reference herein.

### BACKGROUND OF THE INVENTION

Pulse oximetry is a widely accepted noninvasive procedure for measuring the oxygen saturation level of arterial blood, an indicator of a person's oxygen supply. Early detection of a low blood oxygen level is critical in the medical field, for example in critical care and surgical applications, because an insufficient supply of oxygen can result in brain damage and death in a matter of minutes. A typical pulse oximetry system utilizes a sensor applied to a patient's finger. The sensor has an emitter configured with both red and infrared LEDs that project light through the finger to a detector so as to determine the ratio of oxygenated and deoxygenated hemoglobin light absorption. In particular, the detector generates first and second intensity signals responsive to the red and IR wavelengths emitted by the LEDs after absorption by constituents of pulsatile blood flowing within a fleshy medium, such as a finger tip. A pulse oximetry sensor is described in U.S. Pat. No. 6,088,607 titled Low Noise Optical Probe, which is assigned to Masimo Corporation, Irvine, CA and incorporated by reference herein.

Capnography comprises the continuous analysis and recording of carbon dioxide concentrations in the respiratory gases of patients. The device used to measure the CO₂ concentrations is referred to as a capnometer. CO₂ monitoring can be performed on both intubated and non-intubated patients. With non-intubated patients, a nasal cannula is used. Capnography helps to identify situations that can lead to hypoxia if uncorrected. Moreover, it also helps in the swift differential diagnosis of hypoxia before hypoxia can lead to irreversible brain damage. Pulse oximetry is a direct monitor of the oxygenation status of a patient. Capnography, on the other hand, is an indirect monitor that helps in the differential diagnosis of hypoxia so as to enable remedial measures to be taken expeditiously before hypoxia results in an irreversible brain damage.

### SUMMARY OF THE INVENTION

Multiple physiological parameters, combined, provide a more powerful patient condition assessment tool than when any physiological parameter is used by itself. For example, a combination of parameters can provide greater confidence if an alarm condition is occurring. More importantly, such a combination can be used to give an early warning of a slowly deteriorating patient condition as compared to any single parameter threshold, which may not indicate such a condition for many minutes. Conditions such as hypovolemia, hypotension, and airway obstruction may develop slowly over time. A physiological parameter system that combines multiple parameters so as to provide an early warning could have a major effect on the morbidity and mortality outcome in such cases.

Further, a greater emphasis has been put on decreasing the pain level of patients on the ward. Accordingly, patients are often given an IV setup that enables the patient to increase the level of analgesia at will. In certain situations, however, the patient's input must be ignored so as to avoid over medication. Complications from over sedation may include hypotension, tachycardia, bradycardia, hypoventilation and apnea. A physiological parameter system that uses pulse oximetry monitoring of SpO₂ and pulse rate in conjunction with patient controlled analgesia (PCA) can aid in patient safety. Utilization of conventional pulse oximetry in conjunction with PCA, however, can result in the patient being erroneously denied pain medication. Conventional monitors are susceptible to patient motion, which is likely to increase with rising pain. Further, conventional monitors do not provide an indication of output reliability.

Advanced pulse oximetry is motion tolerant and also provides one or more indications of signal quality of data confidence. These indicators can be used as arbitrators in decision algorithms for adjusting the PCA administration and sedation monitoring. Further, advanced pulse oximetry can provide parameters in addition to oxygen saturation and pulse rate, such as perfusion index (PI). For example hypotension can be assessed by changes in PI, which may be associated with changes in pulse rate. Motion tolerant pulse oximetry is described in U.S. Pat. No. 6,699,194 titled Signal Processing Apparatus and Method; signal quality and data confidence indicators are described in U.S. Pat. No. 6,684,090 titled Pulse Oximetry Data Confidence Indicator, both of which are assigned to Masimo Corporation, Irvine, CA and incorporated by reference herein.

One aspect of a physiological parameter system in a first parameter input responsive to a first physiological sensor and a second parameter input responsive to a second physiological sensor. A processor is adapted to combine the parameters and predetermined limits for the parameters so as to generate an alarm output.

Another aspect of a physiological parameter system is a parameter input responsive to a physiological sensor and a quality indicator input relating to confidence in the parameter input. A processor is adapted to combine the parameter input, the quality indicator input and predetermined limits for the parameter input and the quality indicator input so as to generate a control output.

A physiological parameter method comprises the steps of inputting a parameter responsive to a physiological sensor and inputting a quality indicator related to data confidence for the parameter. A control signal is output from the combination of the parameter and the quality indicator. The control signal is adapted to affect the operation of a medical-related device.

### BRIEF DESCRIPTION OF THE DRAWINGS

FIG. 1 is a general block diagram of a physiological parameter system having alarm, diagnostic and control outputs;

FIG. 2 is a block diagram of a physiological parameter system combining pulse oximetry and capnography and providing alarm outputs;

FIG. 3 is a block diagram of a saturation limit alarm enhanced by ETCO₂ measurements;

FIG. 4 is a block diagram of a CO₂ waveform alarm enhanced by SpO₂ measurements;

FIG. 5 is a block diagram of a physiological parameter system combining pulse oximetry and capnography and providing a diagnostic output; and

FIGS. 6A, 6B and 7 are block diagrams of a physiological parameter system utilizing pulse oximetry to control patient controlled analgesia (PCA).

### DETAILED DESCRIPTION OF THE PREFERRED EMBODIMENTS

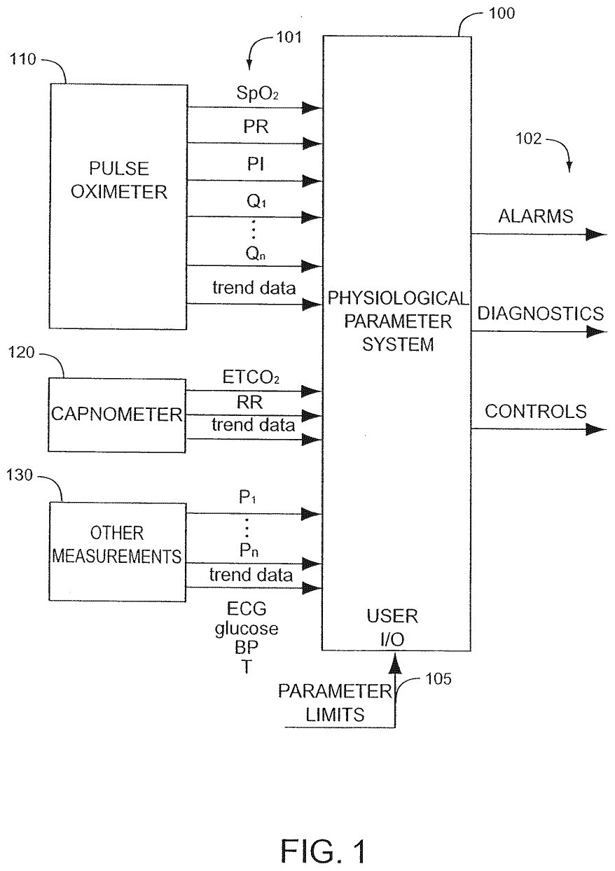

FIG. 1 illustrates a physiological parameter system 100, which may comprise an expert system, a neural-network or a logic circuit, for example. The physiological parameter system 100 has as inputs 101 one or more parameters from one or more physiological measurement devices, such as a pulse oximetry 110 and/or a capnometer 120. Pulse oximeter parameters may include oxygen saturation (SpO₂), perfusion index (PI), pulse rate (PR), various signal quality and/or data confidence indicators (Qn) and trend data, to name a few. Capnography parameter inputs may include, for example, an exhaled carbon dioxide waveform, end tidal carbon dioxide (ETCO₂) and respiration rate (RR). Signal quality and data confidence indicators are described in U.S. Pat. No. 6,684,090 cited above. The physiological parameter system 100 may also have parameter limits 105, which may be user inputs, default conditions or otherwise predetermined thresholds within the system 100.

The inputs 101 are processed in combination to generate one or more outputs 102 comprising alarms, diagnostics and controls. Alarms may be used to alert medical personnel to a deteriorating condition in a patient under their care. Diagnostics may be used to assist medical personnel in determining a patient condition. Controls may be used to affect the operation of a medical-related device. Other measurement parameters 130 that can be input to the monitor may include or relate to one or more of ECG, blood glucose, blood pressure (BP), temperature (T), HbCO and MetHb, to name a few.

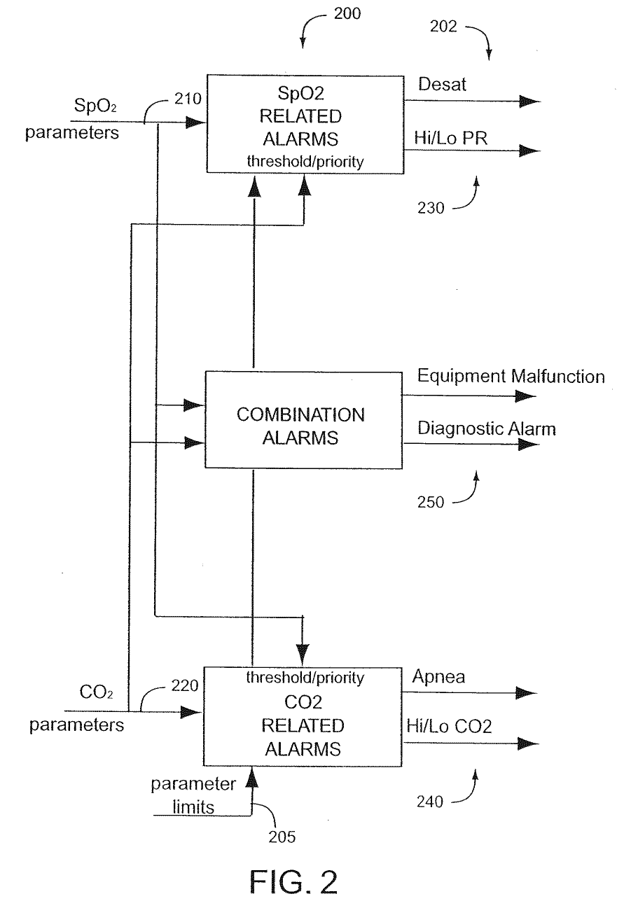

FIG. 2 illustrates one embodiment of a physiological parameter system 200 combining pulse oximetry parameter inputs 210 and capnography parameter inputs 220 so as to generate alarm outputs 202. Parameter limits 205 may be user inputs, default conditions or otherwise predetermined alarm thresholds for these parameters 210, 220. The alarms 202 are grouped as pulse oximetry related 230, capnography related 240 and a combination 250. For example, a pulse oximetry alarm 230 may be related to percent oxygen saturation and trigger when oxygen saturation falls below a predetermined percentage limit. A capnography alarm 240 may be related to ETCO₂ and trigger when ETCO₂ falls below or rises above a predetermined mm Hg pressure limit. A combination alarm 250 may indicate a particular medical condition related to both pulse oximetry and capnography or may indicate a malfunction in either instrument.

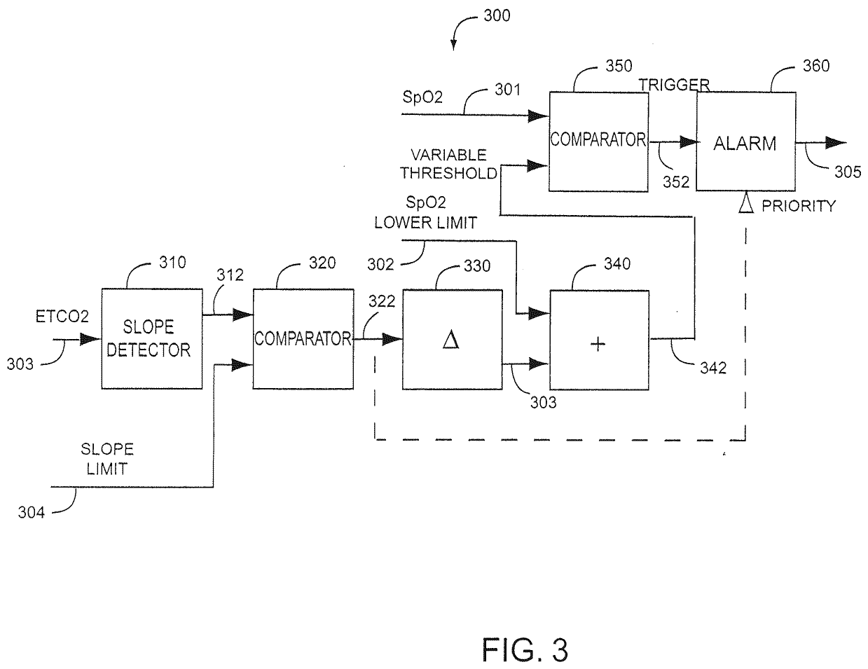

FIG. 3 illustrates a SpO₂ alarm embodiment 300 that is responsive to ETCO₂. In particular, a SpO₂ alarm 305 may be triggered sooner and may indicate a high priority if ETCO₂ 303 is falling. That is, if ETCO₂ 303 is trending down above a certain rate, the SpO₂ alarm 305 is triggered at a higher percentage oxygen saturation threshold and alerts a caregiver to the possibility of a serious condition, e.g., a pulmonary embolism.

As shown in FIG. 3, a slope detector 310 determines the slope 312 of the ETCO₂ input 303. A slope comparator 320 compares this slope 312 to a predetermined slope limit 304. If the downward trend of ETCO₂ 303 is great enough, a delta value 303 is added 340 to the SpO₂ lower limit 302 to generate a variable threshold 342. A threshold comparator 350 compares this variable threshold 342 to the SpO₂ input 301 to generate a trigger 352 for the SpO₂ alarm 305. The alarm volume, modulation or tone may be altered to indicate priority, based upon the slope comparator output 322.

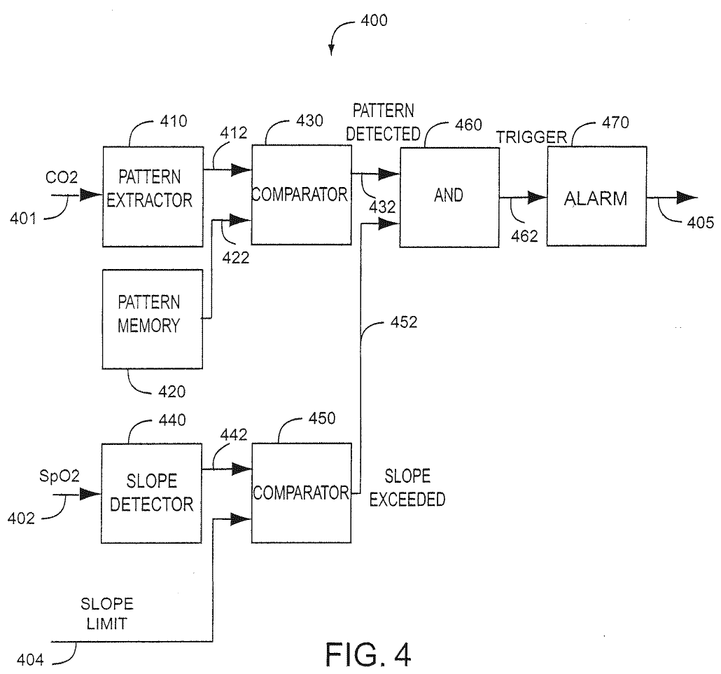

FIG. 4 illustrates a CO₂ alarm embodiment 400 that is responsive to SpO₂. In particular, morphology of the input CO₂ waveform 401 is utilized to trigger an alarm 405, and that alarm is also responsive to a falling SpO₂ 402. That is, if a pattern in the expired CO₂ waveform is detected and SpO₂ is trending down above a certain rate, then an alarm is triggered. For example, an increasing slope of the CO₂ plateau in combination with a downward trend of SpO₂ may trigger an alarm and alert a caregiver to the possibility of an airway obstruction.

As shown in FIG. 4, a pattern extractor 410 identifies salient features in the CO₂ waveform and generates a corresponding feature output 412. A pattern memory 420 stores one or more sets of predetermined waveform features to detect in the CO₂ input 401. The pattern memory 420 is accessed to provide a feature template 422. A feature comparator 430 compares the feature output 412 with the feature template 422 and generates a match output 432 indicating that a specific shape or pattern has been detected in the CO₂ waveform 401. In addition, a slope detector 400 determines the slope 442 of the SpO₂ input 402. A slope comparator 450 compares this slope 442 to a predetermined slope limit 404. If the downward trend of SpO₂ 402 is great enough, a slope exceeded output 452 is generated. If both the match output 432 and the slope exceeded output 452 are each asserted or “true,” then a logical AND 460 generates a trigger output 462 to the alarm 470, which generates an alarm output 405.

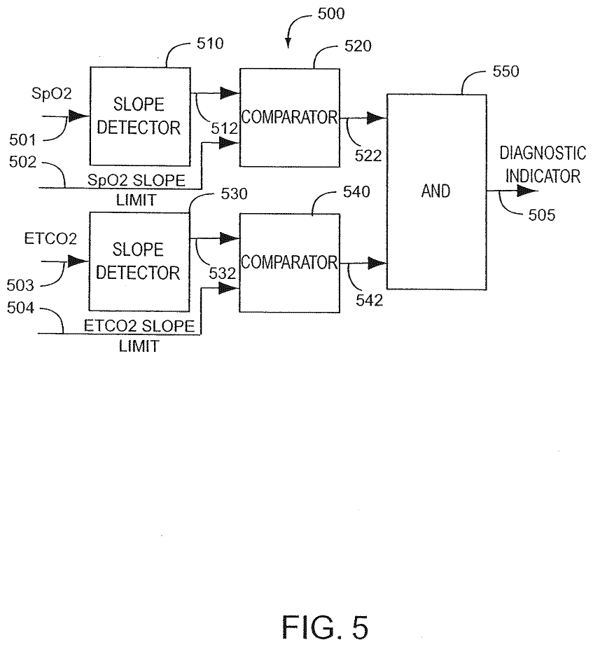

FIG. 5 illustrates a combination embodiment 500 having a diagnostic output 505 responsive to both SpO₂ 501 and ETCO₂ 503 inputs. A SpO₂ slope detector 510 determines the slope 512 of the SpO₂ input 501 and can be made responsive to a negative slope, a positive slope or s slope absolute value. A first comparator 520 compares this slope 512 to a predetermined SpO₂ slope limit 502. If the trend of SpO₂ 501 is great enough, a SpO₂ slope exceeded output 522 is asserted. Likewise, an ETCO₂ slope detector 530 determines the slope 532 of the ETCO₂ input 503. A second comparator 540 compares this slope 532 to a predetermined ETCO₂ slope limit 504. If the downward trend of ETCO₂ 501 is great enough, an ETCO₂ slope exceeded output 542 is asserted. If both slope exceeded outputs 522, 542 are asserted or “true,” a diagnostic output 505 is asserted.

In one embodiment, the slope detectors 510, 530 are responsive to a negative trend in the SpO₂ 501 and ETCO₂ 503 inputs, respectively. Accordingly, the diagnostic output 505 indicates a potential embolism or cardiac arrest. In another embodiment, the SpO₂ slope detector 510 is responsive to negative trends in the SpO₂ 501 input, and the ETCO₂ slope detector 530 is responsive to a positive trend in the ETCO₂ 503 input. Accordingly, the diagnostic output 505 indicates a potential airway obstruction. The diagnostic output 505 can trigger an alarm, initiate a display, or signal a nursing station, to name a few.

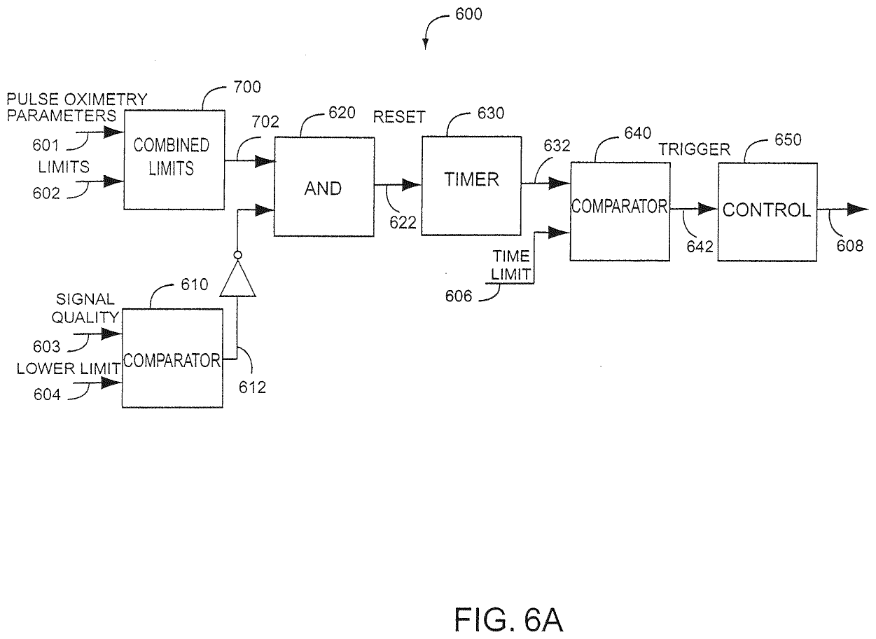

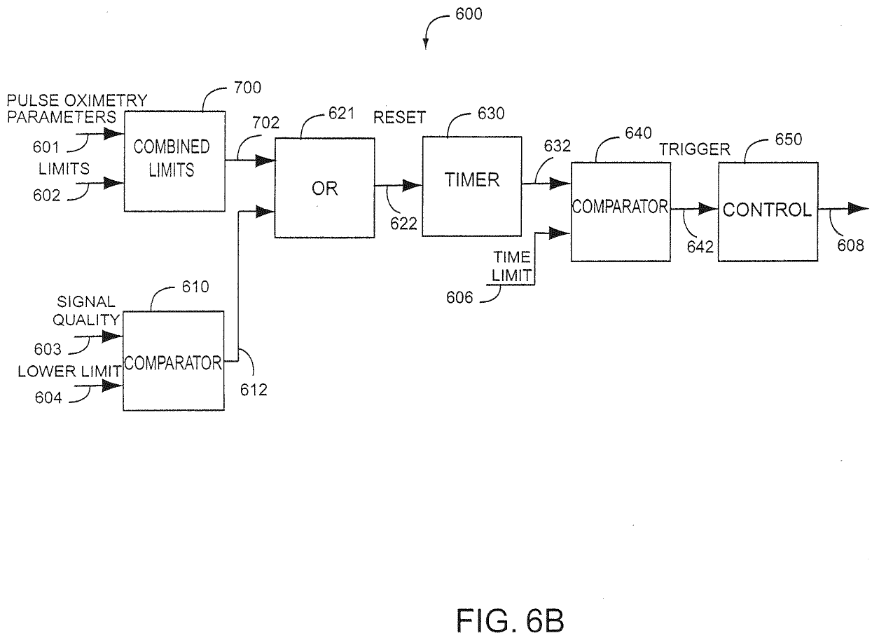

FIGS. 6A-B illustrate a physiological parameter system 600 utilizing pulse oximetry to control patient controlled analgesia (PCA). In particular embodiments, a control output 608 is responsive to pulse oximetry parameters 601 only if signal quality 603 is above a predetermined threshold 604. In FIG. 6A, the control output 608 can be used to lock-out patient controlled analgesia (PCA) if pulse oximetry parameter limits have been exceeded. If signal quality is so low that those parameters are unreliable, however, PCA is advantageously allowed. That is, the pulse oximetry parameters are not allowed to lock-out PCA if those parameters are unreliable. By contrast, in FIG. 6B, the control output 608 can be used to advantageously lock-out or disable patient controlled analgesia (PCA) if pulse oximetry parameter limits have been exceeded or if signal quality is so low that those parameters are unreliable.

As shown in FIG. 6A, pulse oximetry parameters 601 and corresponding limits 602 for those parameters are one set of inputs and a signal quality measures 603 and a corresponding lower limit 604 for signal quality are another set of inputs. The parameters 601 and corresponding limits 602 generate a combined output 703 that is asserted if any of the pulse oximetry parameter limits are exceeded. A comparator 610 compares the signal quality 603 input with a lower limit 604 generating a quality output 612 that is asserted if the signal quality 603 drops below that limit 604. An AND logic 620 generates a reset 622 if the combined output 702 is asserted and the quality output 612 is not asserted. The reset 622 resets the timer 630 to zero. A comparator 640 compares the timer output 632 to a predetermined time limit 606 and generates a trigger 642 if the time limit is exceeded. The trigger 642 causes the control 650 to generate the control output 608, enabling a patient controlled analgesia (PCA), for example. In this manner, the PCA is enabled if all monitored parameters are within set limits and signal quality is above its lower limit for a predetermined period of time.

As shown in FIG. 6B, the combined output 702, quality output 612, reset 622, timer 630, comparator 640 and control 650 are generated as described with respect to FIG. 6A, above. An OR logic 621 generates a rest 622 if either the combined output 702 or the quality output 612 is asserted. In this manner, the PCA is disabled for a predetermined period of time if any of the monitored parameters are outside of set limits or the signal quality is below its lower limit.

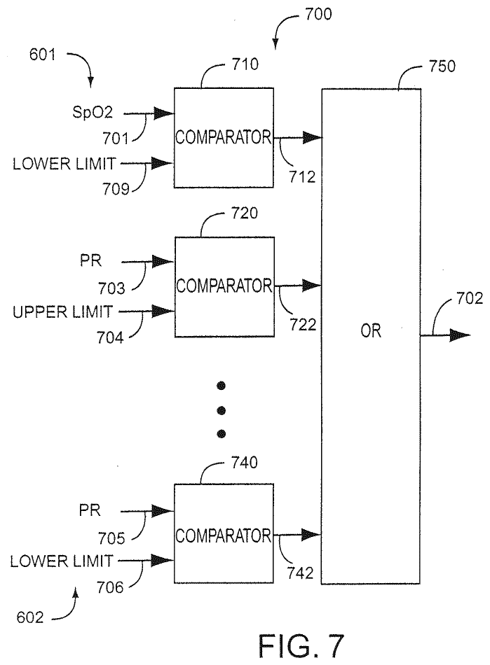

FIG. 7 illustrates combined limits 700 having SpO₂ parameters 601 and corresponding thresholds 602 as inputs and providing a combination output 702. In particular, if any parameter 601 exceeds its corresponding limit 602, the output of the corresponding comparator 710, 720, 740 is asserted. An OR logic 750 is responsive to any asserted output 712, 722, 742 to asserted the combined output 702. For example, the combined output 702 may be asserted if SpO₂ 701 falls below a lower limit 709, pulse rate (PR) 703 rises above an upper limit 704 or PR 703 falls below a lower limit 706.

A physiological parameter system has been disclosed in detail in connection with various embodiments. These embodiments are disclosed by way of examples only and are not to limit the scope of the claims that follow. One of ordinary skill in the art will appreciate many variations and modifications. For example, the control output 608 (FIG. 6B) can be used to control (titrate) delivered, inspired oxygen levels to patients based upon pulse oximetry parameters, unless signal quality is so low that those parameters are unreliable. One of ordinary skill in the art will also recognize that the control output 608 (FIG. 6B) can be used to control patient delivery of any of various pharmacological agents and/or medical gases.

What is claimed is:

1. A patient monitor comprising:
a first input configured to receive a first parameter signal, said first parameter signal indicative of a first physiological parameter of a patient and said first parameter signal responsive to a first physiological sensor monitoring said patient;
a second input configured to receive a second parameter signal, the second parameter signal indicative of a second physiological parameter of the patient and responsive to a second physiological sensor monitoring said patient;
a processor responsive to said first and second inputs to determine trend data based on one or more of the first parameter signal and the second parameter signals and, to generate an alarm output based at least in part on said trend data; and
a pattern detector having a detection output responsive to said first parameter signal,
said diagnostic indication of a patient condition or alarm output responsive to said detection output and said trend data.

2. The patient monitor according to claim 1 further comprising:
a slope detector output responsive to said second parameter signal to generate said trend data; and
a predetermined slope limit responsive to said slope detector output,
wherein said diagnostic indication of a patient condition or alarm output is triggered only if said slope detector output exceeds said slope limit.

3. The patient monitor according to claim 2 wherein:
said first parameter signal comprises values responsive to an ETCO₂ of the patient;
said second parameter signal comprises values responsive to an SpO₂ of the patient; and
said diagnostic indication of a patient condition or alarm output is responsive to ETCO₂ morphology only when there is a sufficient downward trend in SpO₂.

4. A physiological parameter system comprising:
a trend data input relating to trend data of a parameter, the parameter responsive to a physiological sensor;
a quality indicator input relating to confidence in said trend data input;
a plurality of predetermined limits for said trend data input and said quality indicator input; and
a processor adapted to combine said inputs and said limits so as to generate a control output.

5. The physiological parameter system according to claim 4 wherein said control output disables patient controlled analgesia when confidence in said trend data input is low.

6. The physiological parameter system according to claim 5 wherein said control output prevents said trend data input from disabling patient controlled analgesia when said quality indicator indicates confidence is low.

7. A physiological parameter method comprising the steps of:
inputting trend data related to a parameter, the parameter responsive to a physiological sensor;
inputting a quality indicator related to data confidence for said trend data;
outputting a control signal from the combination of said trend data and said quality indicator,
wherein said control signal is adapted to affect the operation of a medical-related device.

8. The physiological parameter method according to claim 7 wherein said trend data and said quality indicator are derived from a pulse oximetry sensor, said outputting step comprising the substeps of:
configuring said control signal to conditionally disable said medical-related device;
regulating said control signal in response to said quality indicator.

9. The physiological parameter method according to claim 8 wherein said regulating substep comprises the substep of disabling a shut-off signal when confidence in said trend data is low.

10. The physiological parameter method according to claim 9 wherein said regulating substep comprises the substep of enabling a shut-off signal when confidence in said trend data is high.
